(19) United States
(12) Patent Application Publication
(10) Pub. No.: US 2023/0094871 A1
(43) Pub. Date: Mar. 30, 2023

(54) ANTI-BACKDRIVE MECHANISM FOR VESSEL SEALING INSTRUMENT

(71) Applicant: Covidien LP, Mansfield, MA (US)

(72) Inventors: Grant T. Sims, Boulder, CO (US); William R. Whitney, Boulder, CO (US); Jason G. Weihe, Longmont, CO (US); James D. Allen, IV, Broomfield, CO (US); Jason T. Sanders, Longmont, CO (US); Alyssa M. Sawyer, Broomfield, CO (US); Curtis M. Siebenaller, Frederick, CO (US)

(21) Appl. No.: 17/953,559

(22) Filed: Sep. 27, 2022

Related U.S. Application Data
(60) Provisional application No. 63/250,376, filed on Sep. 30, 2021.

(51) Int. Cl. A61B 18/14

(52) U.S. Cl. CPC A61B 18/1447; A61B 2018/1455; A61B 2018/0063

(57) ABSTRACT

A vessel sealing instrument includes a housing having a shaft extending from a distal end thereof including an end effector assembly having opposing first and second jaw members operably coupled thereto. The jaw members movable between open and closed positions for clamping tissue with a closure pressure. The jaw members are adapted to connect to a generator for providing energy thereto. An anti-backdrive mechanism is operably associated with the end effector assembly, the anti-backdrive mechanism including first and second mesh-like electrodes disposed in opposing relation on respective first and second jaw members and including openings defined therein. The first and second mesh-like electrodes compressible between a first configuration for grasping tissue wherein the openings defined within each mesh-like electrode include a first diameter to a second configuration for sealing tissue wherein the openings defined within each mesh-like electrode expand to a second diameter configured to release steam upon activation.

### CROSS REFERENCE TO RELATED APPLICATION

This application claims the benefit of U.S. Provisional Application Ser. No. 63/250,376 filed Sep. 30, 2021, the entire contents of which being incorporated by reference herein.

### FIELD

The present disclosure relates to surgical instruments and, more particularly, to anti-backdrive mechanisms for vessel sealing instruments configured to maintain closure pressure during sealing.

### BACKGROUND

A surgical forceps is a pliers-like surgical instrument that relies on mechanical action between its jaw members to grasp, clamp, and constrict tissue. Electrosurgical forceps utilize both mechanical clamping action and energy to heat tissue to treat, e.g., coagulate, cauterize, or seal, tissue. Typically, once tissue is grasped under a closure pressure suitable to seal vessels or tissue, the actuation mechanism (e.g., handle) is locked during the delivery of electrosurgical energy to produce a seal. In some instance the surgeon holds the actuation mechanism during electrosurgical activation. During sealing, the tissue naturally expands against the closure pressure which, in some instances, can affect the resulting tissue seal as the closure pressure no longer falls within a particular closure pressure range.

Accordingly, there exists a need to maintain the closure pressure within the desired closure pressure range during the sealing process.

### SUMMARY

As used herein, the term “distal” refers to the portion that is being described which is further from a user, while the term “proximal” refers to the portion that is being described which is closer to a user. Further, to the extent consistent, any or all of the aspects detailed herein may be used in conjunction with any or all of the other aspects detailed herein.

Provided in accordance with aspects of the present disclosure is a vessel sealing instrument including a housing having a shaft extending from a distal end thereof. A distal end of the shaft includes an end effector assembly having a pair of opposing first and second jaw members operably coupled thereto. One or both of the first or second jaw members is movable between an open position and a closed position for clamping tissue with a closure pressure within the range of about 3 kg/cm² to about 16 kg/cm². One or both of the first or second jaw members is adapted to connect to a generator configured to provide electrosurgical energy thereto in accordance with a sealing algorithm upon activation thereof.

An anti-backdrive mechanism is operably associated with the end effector assembly and includes first and second mesh-like electrodes disposed in opposing relation on respective first and second jaw members. The first and second mesh-like electrodes include openings defined therein. The first and second mesh-like electrodes are compressible between a first configuration for grasping tissue under a grasping pressure wherein the openings defined within each mesh-like electrode include a first size to a second configuration for sealing tissue within the closure pressure wherein the openings defined within each mesh-like electrode expand to a second size configured to release steam upon activation of the mesh-like electrode to seal tissue.

In aspects according to the present disclosure, upon return of the first and second jaw members to the open position, the mesh-like electrodes return to the first configuration. In other aspects according to the present disclosure, upon return of the first and second jaw members to the open position, the mesh-like electrodes return to the first configuration under a spring bias.

In aspects according to the present disclosure, electrosurgical energy is provided to the mesh-like electrodes when the mesh-like electrodes are disposed in the second configuration. In aspects according to the present disclosure, electrosurgical energy is provided to the mesh-like electrodes prior to the mesh-like electrodes transitioning to the second configuration.

In aspects according to the present disclosure, in the first configuration a grasping area is defined between the mesh-like electrodes for initially grasping tissue and wherein during transition to the second configuration, the mesh-like electrodes expand along the tissue to form a larger area therebetween for sealing tissue.

In aspects according to the present disclosure, a gap defined between the mesh-like electrodes when disposed in the second configuration is within the range of about 0.001 inches to about 0.006 inches. In other aspects according to the present disclosure, the gap defined between the mesh-like electrodes during sealing is maintained within the range of about 0.001 inches to about 0.006 inches. In still other aspects according to the present disclosure, the gap defined between the mesh-like electrodes remains the same during sealing.

Provided in accordance with aspects of the present disclosure is a vessel sealing instrument including a housing having a shaft extending from a distal end thereof. A distal end of the shaft includes an end effector assembly having a pair of opposing first and second jaw members operably coupled thereto. One or both of the first or second jaw members is movable between an open position wherein the jaw members are spaced relative to one another and a closed position for clamping tissue with a closure pressure within the range of about 3 kg/cm² to about 16 kg/cm² and a gap distance defined between the jaw members of about 0.001 inches to about 0.006 inches. One or both of the first or second jaw members is adapted to connect to a generator configured to provide electrosurgical energy thereto in accordance with a sealing algorithm upon activation thereof.

An anti-backdrive mechanism is operably associated with the end effector assembly, and includes first and second mesh-like electrodes disposed in opposing relation on respective first and second jaw members. The first and second mesh-like electrodes including openings defined therein. The first and second mesh-like electrodes are compressible between a first configuration for grasping tissue under a grasping pressure wherein the openings defined within each mesh-like electrode include a first shape, e.g., diameter, to a second configuration for sealing tissue within the closure pressure wherein the openings defined within each mesh-like electrode expand to a second shape, e.g., diameter, configured to release steam upon activation of the mesh-like electrode to maintain the jaw members within the gap distance for sealing tissue.

In aspects according to the present disclosure, the orientation of the mesh may increase seal strength, e.g., relieve stress concentrations and help denature the tissue, e.g., cross-diagonal pattern normal to the tissue.

In aspects according to the present disclosure, upon return of the first and second jaw members to the open position, the mesh-like electrodes return to the first configuration. In other aspects according to the present disclosure, upon return of the first and second jaw members to the open position, the mesh-like electrodes return to the first configuration under a spring bias.

In aspects according to the present disclosure, electrosurgical energy is provided to the mesh-like electrodes when the mesh-like electrodes are disposed in the second configuration. In aspects according to the present disclosure, electrosurgical energy is provided to the mesh-like electrodes prior to the mesh-like electrodes transitioning to the second configuration.

In aspects according to the present disclosure, in the first configuration a grasping area is defined between the mesh-like electrodes for initially grasping tissue and wherein during transition to the second configuration, the mesh-like electrodes expand along the tissue to form a larger area therebetween for sealing tissue.

In aspects according to the present disclosure, wherein the gap defined between the mesh-like electrodes during sealing is maintained within the range of about 0.001 inches to about 0.006 inches. In other aspects according to the present disclosure, the gap defined between the mesh-like electrodes remains the same during sealing.

### BRIEF DESCRIPTION OF DRAWINGS

The above and other aspects and features of the present disclosure will become more apparent in view of the following detailed description when taken in conjunction with the accompanying drawings wherein like reference numerals identify similar or identical elements.

FIG. 1A is a perspective view of an electrosurgical forceps provided in accordance with the present disclosure having in-line electrosurgical activation;

FIG. 1B is a perspective view of an electrosurgical forceps provided in accordance with another embodiment of the present disclosure having a ratchet-like handle assembly;

FIG. 2A is an enlarged, perspective view of an end effector assembly of the electrosurgical forceps of FIG. 1 wherein first and second jaw members of the end effector assembly are disposed in a spaced-apart position;

FIG. 2B is an enlarged, perspective view of the end effector assembly of FIG. 2A wherein the first and second jaw members are disposed in an approximated position;

FIG. 3A is a side view of a proximal portion of the electrosurgical forceps of FIG. 1 with a movable handle and trigger thereof disposed in respective un-actuated positions;

FIG. 3B is a side view of the proximal portion of the electrosurgical forceps shown in FIG. 3A with the movable handle disposed in an actuated position and the trigger disposed in the un-actuated position;

FIG. 3C is a side view of the proximal portion of the electrosurgical forceps shown in FIG. 3A with the movable handle and trigger disposed in respective actuated positions;

FIG. 4A is a side view of another proximal portion of the electrosurgical forceps of FIG. 1 with portions removed to illustrate a trigger assembly thereof with the trigger disposed in the un-actuated position;

FIG. 4B is a side view of the proximal portion of the electrosurgical forceps shown in FIG. 4A with portions removed to illustrate the trigger assembly with the trigger disposed in the actuated position;

FIG. 5A is an enlarged, side view of an end effector assembly having mesh-like electrically conductive sealing plates on the opposing first and second jaw members, the forceps shown grasping tissue;

FIG. 5B is a greatly-enlarged view of the area of detail of FIG. 5A;

FIG. 5C is a view of the end effector assembly of FIG. 5A with the opposing first and second jaw members compressed during tissue sealing; and

FIG. 5D is a greatly-enlarged view of the area of detail of FIG. 5C

### DETAILED DESCRIPTION

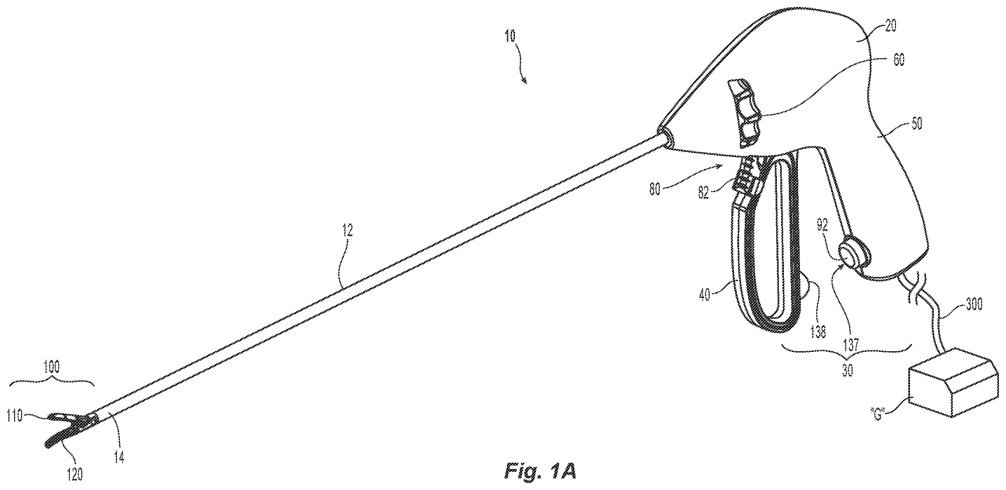

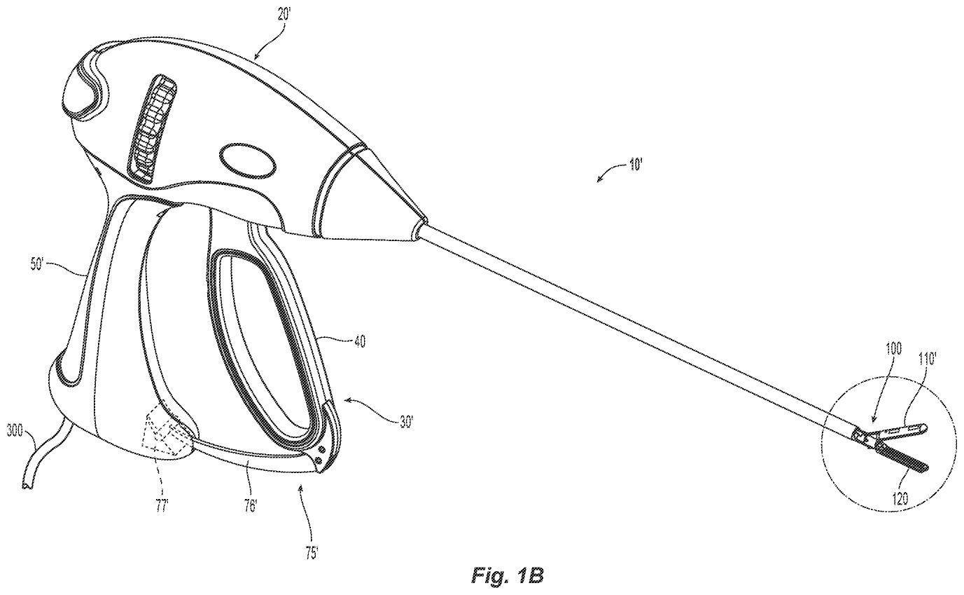

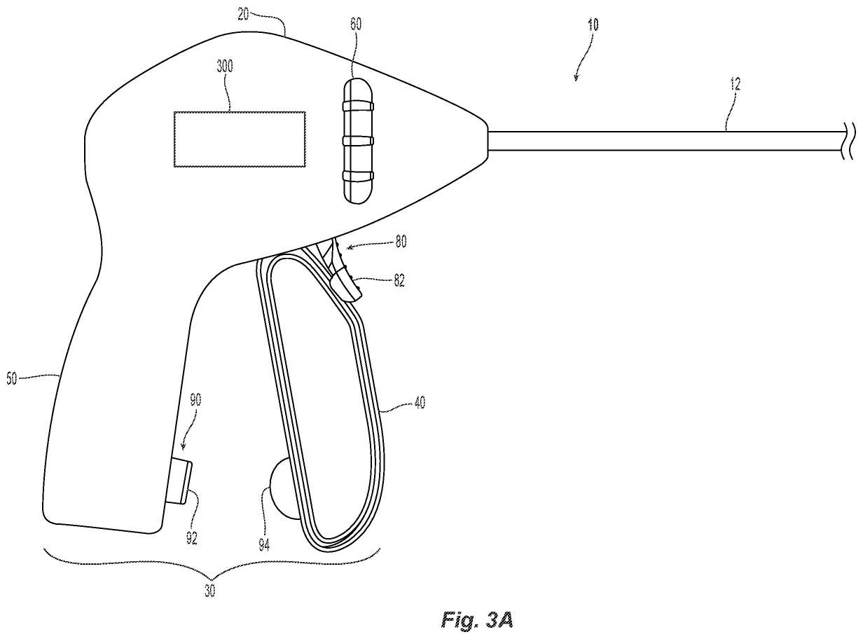

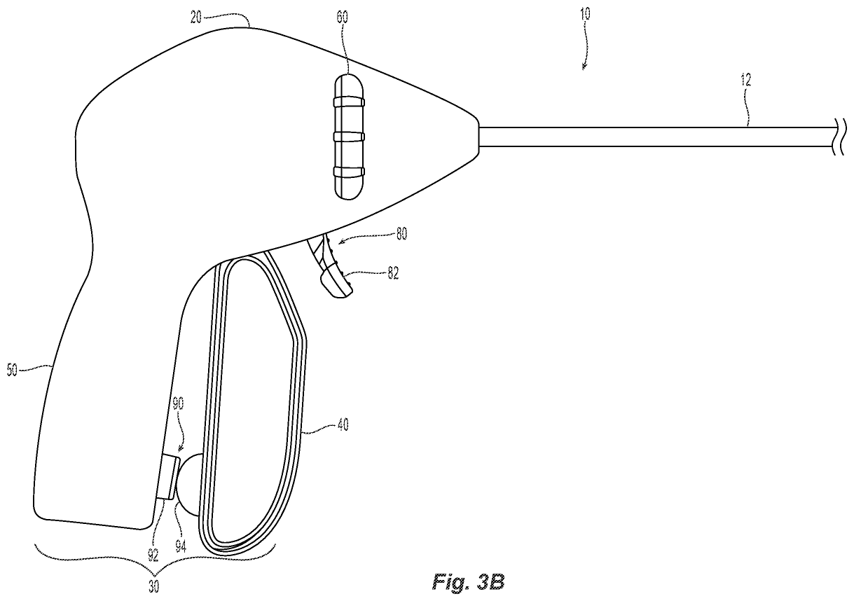

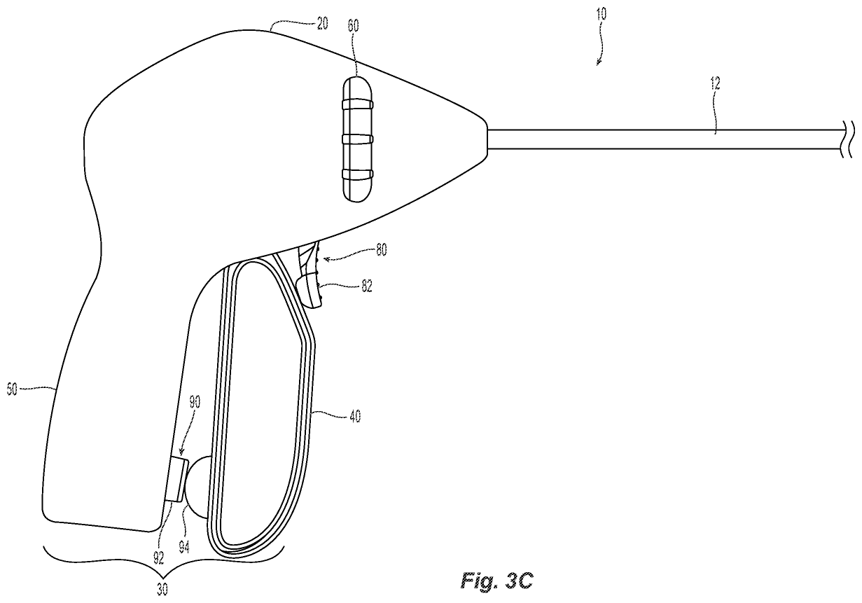

Referring to FIG. 1A, a surgical instrument provided in accordance with the present disclosure is shown configured as a bipolar electrosurgical forceps 10 for use in connection with endoscopic surgical procedures, although the present disclosure may be equally applicable for use with other surgical instruments such as those for use in endoscopic and/or traditional open surgical procedures. Forceps 10 generally includes a housing 20, a handle assembly 30, a rotating assembly 60, a trigger assembly 80, an activation assembly 90 (See FIGS. 3A-3C), and an end effector assembly 100 including first and second jaw members 110, 120.

Forceps 10 further includes a shaft 12 having a distal end portion 14 configured to engage (directly or indirectly) end effector assembly 100 and a proximal end portion 16 that engages (directly or indirectly) housing 20. Rotating assembly 60 is rotatable in either direction to rotate shaft 12 and end effector assembly 100 relative to housing 20 in either direction. Housing 20 houses the internal working components of forceps 10.

An electrosurgical cable 300 connects forceps 10 to an electrosurgical generator “G” or other suitable energy source, although forceps 10 may alternatively be configured as a handheld instrument incorporating energy-generating and/or power components thereon or therein. Cable 300 includes wires (not shown) extending therethrough, into housing 20, and through shaft 12, to ultimately connect electrosurgical generator “G” to jaw member 110 and/or jaw member 120 of end effector assembly 100. Activation button 92 of activation assembly 90 is disposed on housing 20 are electrically coupled between end effector assembly 100 and cable 300 to enable the selective supply of energy to jaw member 110 and/or jaw member 120, e.g., upon activation of activation button 92. However, other suitable electrical connections and/or configurations for supplying electrosurgical energy to jaw member 110 and/or jaw member 120 may alternatively be provided, as may other suitable forms of energy, e.g., ultrasonic energy, microwave energy, light energy, thermal energy, etc.

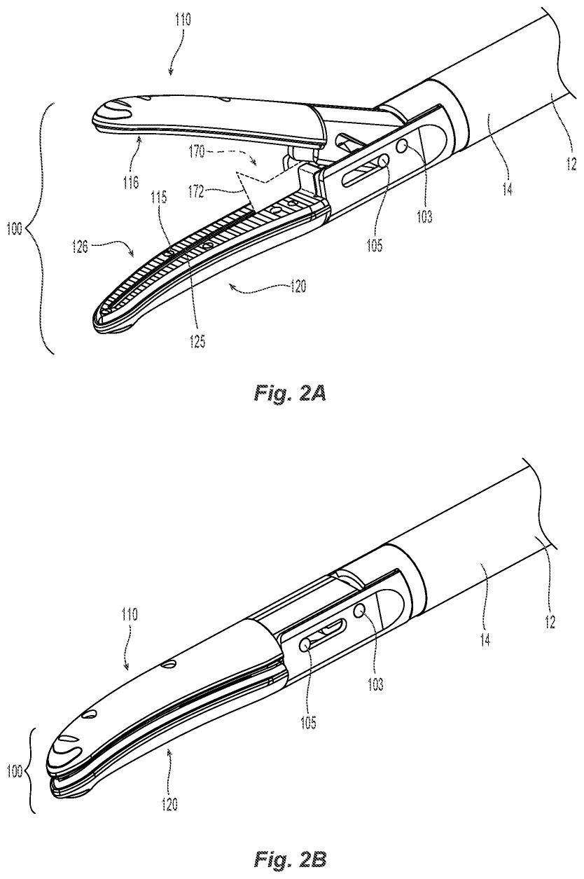

Forceps 10 additionally includes a knife assembly 170 (FIG. 2A) operably coupled to trigger assembly 80 and extending through housing 20 and shaft 12. One or both of jaw members 110, 120 defines a knife channel 125 (FIG. 2A) configured to permit reciprocation of a knife blade 172 (FIG. 2A) of knife assembly 170 (FIG. 2A) therethrough, e.g., in response to actuation of trigger 82 of trigger assembly 80. Trigger assembly 80 is described in greater detail below as are other embodiments of trigger assemblies configured for use with forceps 10.

With additional reference to FIGS. 2A and 2B, end effector assembly 100, as noted above, is disposed at distal end portion 14 of shaft 12 and includes a pair of jaw members 110 and 120 pivotable between a spaced-apart position and an approximated position for grasping tissue therebetween. End effector assembly 100 is designed as a unilateral assembly, e.g., wherein one of the jaw members 120 is fixed relative to shaft 12 and the other jaw member 110 is movable relative to both shaft 12 and the fixed jaw member 120. However, end effector assembly 100 may alternatively be configured as a bilateral assembly, e.g., wherein both jaw member 110 and jaw member 120 are movable relative to one another and with respect to shaft 12.

Each jaw member 110, 120 of end effector assembly 100 includes an electrically-conductive tissue-contacting surface 116, 126. Tissue-contacting surfaces 116 are positioned to oppose one another for grasping and treating tissue. More specifically, tissue-contacting surfaces 116, 126 are electrically coupled to the generator “G,” e.g., via cable 300, and activation button 92 to enable the selective supply of energy thereto for conduction through tissue grasped therebetween, e.g., upon activation of activation button 92. One or both of tissue-contacting surfaces 116, 126 may include one or more stop members 115 extending therefrom to define a minimum gap distance between electrically-conductive tissue-contacting surfaces 116, 126 in the approximated position of jaw members 110, 120, facilitate grasping of tissue, and/or inhibit shorting between electrically-conductive tissue-contacting surfaces 116, 126.

The stop member(s) 115 may be formed at least partially from an electrically-insulative material or may be effectively insulative by electrically isolating the stop member(s) from one or both of the electrically-conductive tissue-contacting surfaces 116, 126. The one or more stop members 115 may be disposed on one or both jaw members 110, 120 or on the tissue-contacting surfaces 116, 126 and are configured to regulate the distance therebetween. Details relating to various stop member designs are disclosed in U.S. Pat. No. 7,857,812, 10,687,887 the entire contents of each of which being incorporated by reference here. Ranges between about 0.001 inches to about 0.006 inches are contemplated.

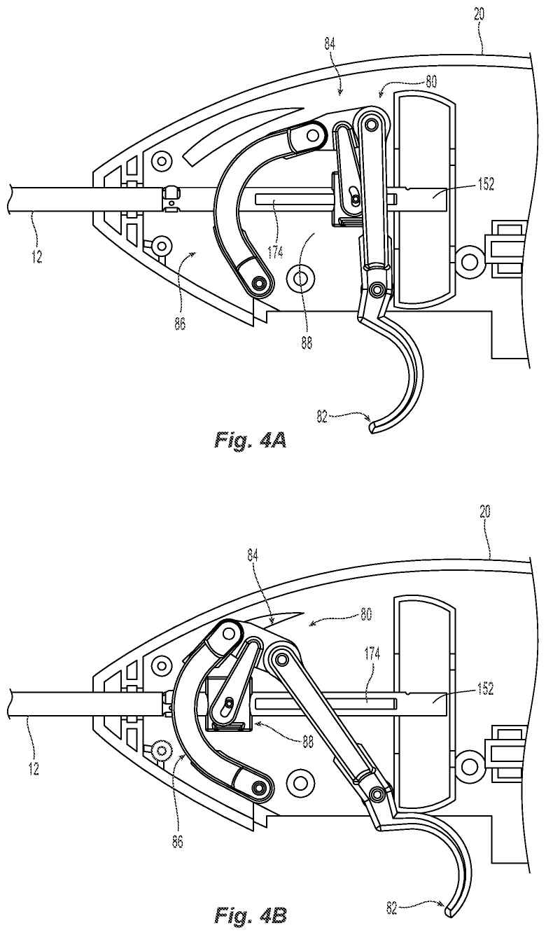

A pivot pin 103 of end effector assembly 100 extends transversely through aligned apertures defined within jaw members 110, 120 and shaft 12 to pivotably couple jaw member 110 to jaw member 120 and shaft 12. A cam pin 105 of end effector assembly 100 extends transversely through cam slots defined within jaw members 110, 120 and is operably engaged with a distal end portion of a drive bar 152 (FIGS. 4A and 4B) of a drive assembly 301 (only drive bar 152 (FIGS. 4A and 4B) of the drive assembly 301 is shown and drive assembly 301 is generically represent by component 301 in FIG. 3A) such that longitudinal translation of drive bar 152 (FIGS. 4A and 4B) through shaft 12 translates cam pin 105 relative to jaw members 110, 120. Various drive assemblies are shown and described with respect to commonly-owned U.S. Pat. Nos. 7,857,812, 8,540,711, 7,384,420, 7,090,673, 7,101,372, 7,255,697, 7,101,371, 7,131,971, 7,083,618, and 10,842,553, the entire contents of each of which being incorporated by reference herein.

More specifically, distal translation of cam pin 105 relative to jaw members 110, 120 urges cam pin 105 distally through the cam slots to thereby pivot jaw members 110, 120 from the spaced-apart position towards the approximated position, although cam slots may alternatively be configured such that proximal translation of cam pin 105 pivots jaw members 110, 120 from the spaced-apart position towards the approximated position. One suitable drive assembly is described in greater detail, for example, in U.S. Pat. No. 9,655,673, the entire contents of which are hereby incorporated herein by reference.

Referring to FIGS. 1A-3C, handle assembly 30 includes a fixed handle 50 and an actuator, e.g., movable handle 40. Fixed handle 50 is integrally associated with housing 20 and movable handle 40 is movable relative to fixed handle 50. Movable handle 40 is ultimately connected to the drive assembly (not shown) that, together, mechanically cooperate to impart movement of jaw members 110 and 120 between the spaced-apart and approximated positions to grasp tissue between electrically-conductive surfaces 116, 126, respectively. More specifically, pivoting of movable handle 40 relative to fixed handle 50 from an un-actuated position towards an actuated position pivots jaw members 110, 120 from the spaced-apart position towards the approximated position. On the other hand, when movable handle 40 is released or returned towards the initial position relative to fixed handle 50, jaw members 110, 120 are returned towards the spaced-apart position.

A biasing spring (not shown) associated with movable handle 40 and/or the drive assembly may be provided to bias jaw members 110, 120 towards a desired position, e.g., the spaced-apart position or the approximated position. Various drive assemblies are shown and described in any one of the above-identified commonly-owned U.S. Patents referenced herein.

Fixed handle 50 operably supports activation button 92 of activation assembly 90 thereon in an in-line position, wherein activation button 92 is disposed in the actuation path of movable handle 40. In this manner, upon pivoting of movable handle 40 relative to fixed handle 50 from the actuated position to an activated position, protrusion 94 of movable handle 40 is urged into contact with activation button 92 to thereby activate activation button 92 and initiate the supply of energy to electrically-conductive surfaces 116, 126, e.g., to treat tissue grasped therebetween. Alternatively, actuation button 92 may be disposed in any other suitable position, on housing 20 or remote therefrom, to facilitate manual activation by a user to initiate the supply of energy to electrically-conductive surfaces 116, 126.

With reference to FIGS. 1A-2B and 4A-4B, as noted above, trigger assembly 80 is operably coupled to knife blade 172 of knife assembly 170. More specifically, trigger 82 of trigger assembly 80 is selectively actuatable, e.g., from an un-actuated position (FIGS. 3A and 4A) to an actuated position (FIGS. 3C and 4B), to deploy knife blade 172 distally through jaw members 110, 120 to cut tissue grasped between electrically-conductive surfaces 116, 126. Knife assembly 170 includes knife blade 172 and a knife bar 174 engaged with and extending proximally from knife blade 172 through shaft 12 and drive bar 152 into housing 20 where knife bar 174 is operably coupled with trigger assembly 80, as detailed below.

Referring to FIGS. 4A and 4B, trigger assembly 80 includes trigger 82, a link 84, e.g., a T-link 84, a link 86, e.g., an arcuate linkage 86 although other configurations, e.g., linear, angled, etc. are also contemplated, and a slider block 88. In this manner, trigger assembly 80 defines a four-bar mechanical linkage assembly for driving slider block 88 to actuate the knife blade 172. This and other types of trigger mechanism are also contemplated such as, for example, trigger mechanisms described in any one of the above-identified commonly-owned U.S. Patents referenced herein or U.S. patent application Ser. No. 16/558,477, the entire contents of each of which being incorporated by reference herein.

As mentioned above, pivoting of movable handle 40 relative to fixed handle 50 from an un-actuated position towards an actuated position pivots jaw members 110, 120 from the spaced-apart position towards the approximated position for grasping tissue therebetween. When fully grasped, the drive assembly 301 is configured to initially generate a closure pressure suitable for sealing vessels upon activation of electrosurgical energy from generator “G”. Maintaining closure pressures within the range of about 3 Kg/cm² to about 16 Kg/cm² are known to promote quality seals.

With in-line actuation instruments, the surgeon is typically required to maintain the handle 40 in position to continually maintain the closure pressure. For example and as shown in FIG. 1A, handle 40 is initially actuated under a light pressure to grasp and manipulate tissue prior to sealing as the jaw members 110, 120 may be closed without fully actuating handle 40 relative to handle 50. Once the tissue is properly positioned between jaw members 110, 120, the handle 40 may be fully actuated to close the jaw members 110, 120 about tissue within the above-noted pressure range and simultaneously activate the forceps 10 for sealing. With this type of forceps 10, the surgeon must maintain the handle 40 fully actuated to maintain the initial closure pressure. This is known as in-line activation.

Other forceps e.g., forceps 10′ of FIG. 1B include handle assemblies 30′ (including movable handle 40′ and fixed handle 50′) that have a ratchet-like locking system 75′ affixed to a portion of the housing 20′ or handle assembly 30′ which is configured to lock handle 40′ relative to the fixed handle 50′ to initially generate and maintain the appropriate closure pressures between jaw members 110′, 120′ when locked. Ratchet-like locking system 75′ includes a flange 76′ extending from handle 40′ configured to mechanically engage and lock within a corresponding ramp 77′ (shown in phantom) disposed within handle 50′. Various such forceps and handle assemblies are shown in any one of the above-identified commonly-owned U.S. Patents referenced herein.

After the initial closure pressure within the above-identified range is generated and the jaw members 110, 120 (or 110′, 120′) are clamped on a vessel or on tissue, the forceps 10 (10′) is ready for activation. As mentioned above, during sealing the vessel or tissue expands against the jaw members, e.g., jaw members 110′, 120′, which may reduce the actual closure pressure during formation of the seal. If the closure pressure falls outside of the above-noted range, the seal may not be as effective.

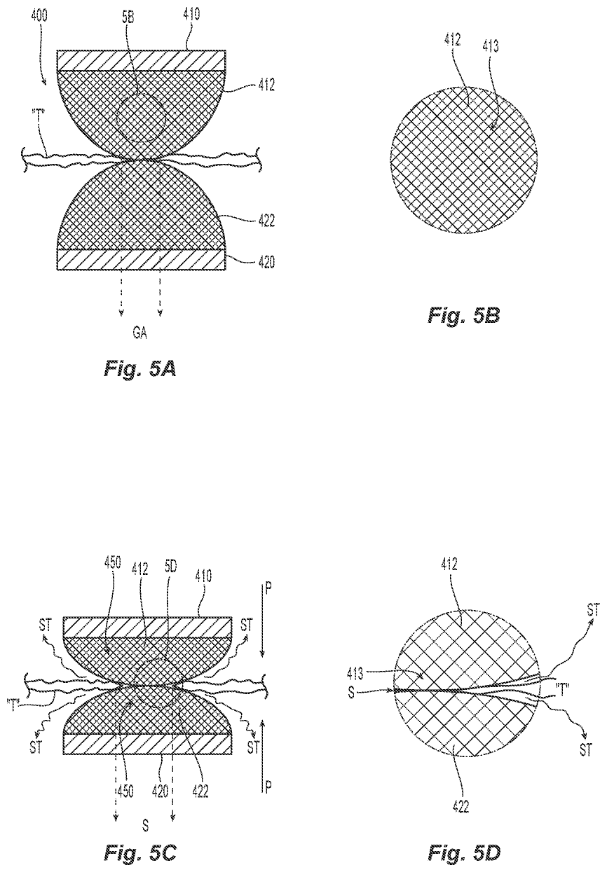

FIGS. 5A-5D show an end effector which includes a so-called “anti-backdrive assembly” configured to maintain the required closure pressure during the sealing process by releasing the build-up of steam between opposing jaw members 110 and 120. The anti-backdrive assembly is configured to maintain the sealing pressure between the jaw members 110, 120 by offloading steam and pressure build-up therebetween normally attributed to tissue expansion. It is envisioned that the below-described anti-backdrive assembly may be characterized as generally passive, e.g., automatically prevents the jaw members from 110, 120 from moving during tissue expansion by virtue of its inherent, steam-releasing design (along with continuous compressibility of mesh).

FIG. 5A shows one embodiment of an end effector assembly 400 having a jaw member 410 equipped with opposing mesh-like electrodes 412, 422 that cooperate as an anti-backdrive assembly 450. More particularly, when is an initial open position (FIG. 1A) or when initially contacting or grasping tissue (FIG. 5A), the opposing mesh-like electrodes 412, 422 include very small or negligible openings 413 defined therein and therebetween which facilitate tissue grasping and manipulation (FIG. 5B). Under the initial, grasping pressure between the mesh-like electrodes, a grasping area “GA” is formed therebetween.

Upon pressure “P” being applied between jaw members 410, 420 to seal tissue “T”, the mesh-like electrodes 412, 422 compress onto the tissue “T” expanding therealong forming a seal area “S” therebetween. In other words, as the jaw members 410, 420 are actuated to compress tissue within the grasping area “GA”, the grasping area “GA” expands eventually forming a larger area “S” for sealing tissue (FIGS. 5C and 5D). Once energized, the mesh-like electrodes 412, 422 act similarly as the conventional sealing electrodes 112, 122 describe above.

As can be appreciated and as described above, during sealing, the tissue “T” melts and reforms into a homogenous fused mass with limited demarcation between opposing tissue structures, e.g., opposing walls of a vessel. Typically, during the sealing process, the tissue “T” expands and lets off steam creating opposing pressure against the closing pressure “P” of the jaw members 410, 420. Additional pressure may be created as the tissue “T” reforms and rehydrates. As mentioned above, it is important to maintain the sealing pressure and gap within the above-identified range during the entire sealing process to insure a proper tissue seal. The backdrive assembly 450 including the mesh-like electrodes 412, 422 of the present disclosure is configured to accomplish this purpose.

More particularly, during closure of the jaw members 410, 420 about tissue “T” the compression of the mesh-like electrodes 412, 422 onto the tissue “T” and the expansion of the openings 413 thereof create an avenue of release for the build-up of steam “ST” along the tissue seal “S” relieving the build-up of pressure between the jaw members 410, 420 and maintaining the jaw members 410, 420 within the appropriate sealing pressure range and gap distance range to form a consistent tissue seal “S”. Upon release of the tissue “T”, the expanded mesh-like electrodes 412, 422 and openings 413 shown in FIG. 5D spring back under a bias or otherwise automatically return to the pre-compressed configuration shown in FIG. 5B.

It is contemplated that the presently designed anti-backdrive assembly 450 may be particularly well-suited for sealing tissue utilizing a so-called “slow close” sealing system, algorithm or methodology wherein when the jaw members 410, 420 are approximated from an open or grasping position to the sealing position (e.g., jaw members 410, 420 closed about tissue within the above-identified gap range) the jaw members 410, 420 are continually energized according to the sealing algorithm. This is in contrast to more traditional tissue sealing wherein the jaw members 410, 420 are fully approximated and then energized to seal tissue.

With the slow close method, the gradual expansion of the openings 413 may be regulated with the closure of the jaw members 410, 420 according to an automated or robotic surgical procedure to insure a consistent and quality tissue seal “S”. This technique may help regulate pressure between the jaw members 410, 420. Various slow close methodologies, systems and algorithms are disclosed in U.S. Pat. No. 8,357,160 entitled “System and Method for Controlling Electrode Gap During Tissue Sealing”, the entire contents of which being incorporated by reference herein.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A vessel sealing instrument, comprising:
a housing having a shaft extending from a distal end thereof, a distal end of the shaft having an end effector assembly including a pair of opposing first and second jaw members operably coupled thereto, at least one of the first or second jaw members movable between an open position and a closed position for clamping tissue with a closure pressure within the range of about 3 kg/cm² to about 16 kg/cm², at least one of the first or second jaw members adapted to connect to a generator configured to provide electrosurgical energy thereto in accordance with a sealing algorithm upon activation thereof; and
an anti-backdrive mechanism operably associated with the end effector assembly, the anti-backdrive mechanism including:
at least one mesh-like electrode disposed in opposing relation to a respective first or second jaw member, the mesh-like electrode including openings defined therein and compressible between a first configuration for grasping tissue under a grasping pressure wherein the openings defined within the mesh-like electrode include a first size to a second configuration for sealing tissue within the closure pressure wherein the openings defined within the mesh-like electrode expand to a second size configured to release steam upon activation of the mesh-like electrode to seal tissue.

2. The vessel sealing instrument according to claim 1, wherein, upon return of the first and second jaw members to the open position, the mesh-like electrode returns to the first configuration.

3. The vessel sealing instrument according to claim 1, wherein, upon return of the first and second jaw members to the open position, the mesh-like electrode returns to the first configuration under a spring bias.

4. The vessel sealing instrument according to claim 1, wherein, electrosurgical energy is provided to the mesh-like electrode when the mesh-like electrode is disposed in the second configuration.

5. The vessel sealing instrument according to claim 1, wherein, electrosurgical energy is provided to the mesh-like electrode prior to the mesh-like electrode transitioning to the second configuration.

6. The vessel sealing instrument according to claim 1, wherein, in the first configuration a grasping area is defined between the mesh-like electrode and the opposing jaw member for initially grasping tissue and wherein during transition to the second configuration, the mesh-like electrode expands along the tissue to form a larger area therebetween for sealing tissue.

7. The vessel sealing instrument according to claim 1, wherein a gap defined between the mesh-like electrode and the opposing jaw member when disposed in the second configuration is within the range of about 0.001 inches to about 0.006 inches.

8. The vessel sealing instrument according to claim 7, wherein the gap defined between the mesh-like electrode and the opposing jaw member during sealing is maintained within the range of about 0.001 inches to about 0.006 inches.

9. The vessel sealing instrument according to claim 7, wherein the gap defined between the mesh-like electrode and the opposing jaw member remains the same during sealing.

10. A vessel sealing instrument, comprising:
a housing having a shaft extending from a distal end thereof, a distal end of the shaft having an end effector assembly including a pair of opposing first and second jaw members operably coupled thereto, at least one of the first or second jaw members movable between an open position wherein the jaw members are spaced relative to one another and a closed position for clamping tissue with a closure pressure within the range of about 3 kg/cm² to about 16 kg/cm² and a gap distance defined between the jaw members of about 0.001 inches to about 0.006 inches, at least one of the first or second jaw members adapted to connect to a generator configured to provide electrosurgical energy thereto in accordance with a sealing algorithm upon activation thereof; and
an anti-backdrive mechanism operably associated with the end effector assembly, the anti-backdrive mechanism including:
first and second mesh-like electrodes disposed in opposing relation on respective first and second jaw members, the first and second mesh-like electrodes including openings defined therein, the first and second mesh-like electrodes compressible between a first configuration for grasping tissue under a grasping pressure wherein the openings defined within each mesh-like electrode include a first diameter to a second configuration for sealing tissue within the closure pressure wherein the openings defined within each mesh-like electrode expand to a second diameter configured to release steam upon activation of the mesh-like electrode to maintain the jaw members within the gap distance for sealing tissue.

11. The vessel sealing instrument according to claim 10, wherein, upon return of the first and second jaw members to the open position, the mesh-like electrodes return to the first configuration.

12. The vessel sealing instrument according to claim 10, wherein, upon return of the first and second jaw members to the open position, the mesh-like electrodes return to the first configuration under a spring bias.

13. The vessel sealing instrument according to claim 10, wherein, electrosurgical energy is provided to the mesh-like electrodes when the mesh-like electrodes are disposed in the second configuration.

14. The vessel sealing instrument according to claim 10, wherein, electrosurgical energy is provided to the mesh-like electrodes prior to the mesh-like electrodes transitioning to the second configuration.

15. The vessel sealing instrument according to claim 10, wherein, in the first configuration a grasping area is defined between the mesh-like electrodes for initially grasping tissue and wherein during transition to the second configuration, the mesh-like electrodes expand along the tissue to form a larger area therebetween for sealing tissue.

16. The vessel sealing instrument according to claim 10, wherein the gap defined between the mesh-like electrodes during sealing is maintained within the range of about 0.001 inches to about 0.006 inches.

17. The vessel sealing instrument according to claim 10, wherein the gap defined between the mesh-like electrodes remains the same during sealing.
